(19) United States
(12) Patent Application Publication
(10) Pub. No.: US 2020/0147275 A1
(43) Pub. Date: May 14, 2020

(54) COPPER BACTERICIDE / VIRUCIDE ASSEMBLY

(71) Applicant: John Ehrhardt, Kingman, AZ (US)

(72) Inventor: John Ehrhardt, Kingman, AZ (US)

(21) Appl. No.: 16/190,618

(22) Filed: Nov. 14, 2018

(51) Int. Cl. A61L 31/02; A61M 31/00; A61L 31/16

(52) U.S. Cl. CPC A61L 31/022; A61M 31/00; A61L 31/16; A61L 2300/404; A61M 2205/0205; A61M 2210/0618; A61L 2300/102; A61M 2202/06

(57) ABSTRACT

A copper bactericide/virucide assembly for killing invasive bacteria and viruses in a nostril includes a wire that is comprised of copper. In this way the wire can kill bacteria and viruses on contact. The wire is insertable into a nostril thereby facilitating the wire to contact mucous membranes within the nostril. Thus, the wire can kill invasive microbes, bacteria and viruses on the mucous membranes thereby reducing the likelihood of developing an upper respiratory infection.

### CROSS-REFERENCE TO RELATED APPLICATIONS

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### BACKGROUND OF THE INVENTION

### (1) Field of the Invention

### (2) Description of Related Art including information disclosed under 37 CFR 1.97 and 1.98

The disclosure and prior art relates to bactericide/virucide devices and more particularly pertains to a new bactericide/virucide device for killing invasive bacteria in a nostril.

### BRIEF SUMMARY OF THE INVENTION

An embodiment of the disclosure meets the needs presented above by generally comprising a wire that is comprised of copper. In this way the wire can kill bacteria and viruses on contact. The wire is insertable into a nostril thereby facilitating the wire to contact mucous membranes within the nostril. Thus, the wire can kill invasive microbes, bacteria and viruses on the mucous membranes thereby reducing the likelihood of developing an upper respiratory infection.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a copper bactericide/virucide assembly according to an embodiment of the disclosure.

FIG. 2 is a top view of an embodiment of the disclosure.

FIG. 3 is a right side view of an embodiment of the disclosure.

FIG. 4 is a perspective in-use view of an embodiment of the disclosure.

FIG. 5 is a perspective view of an alternative embodiment of the disclosure.

### DETAILED DESCRIPTION OF THE INVENTION

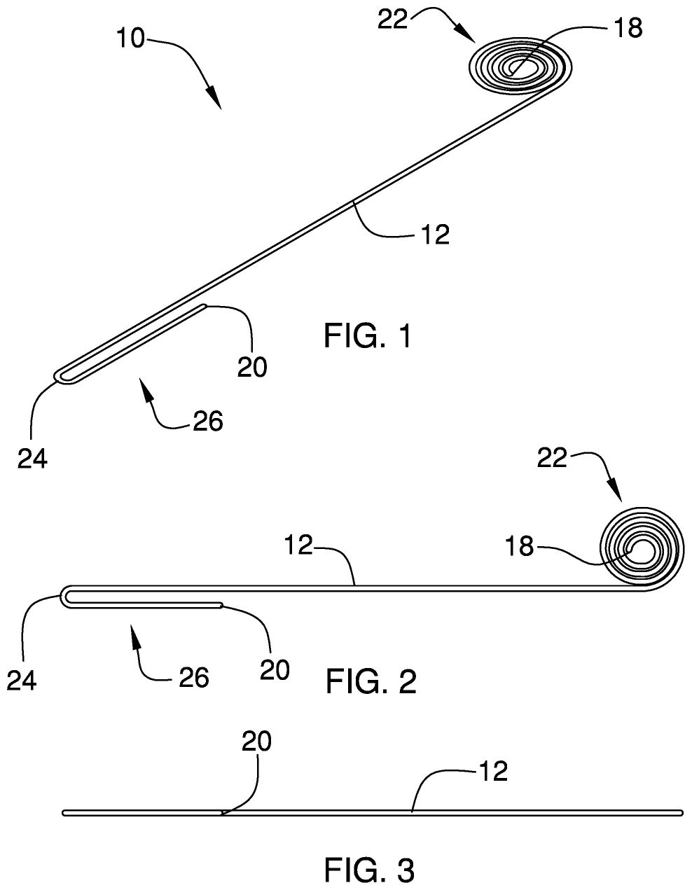

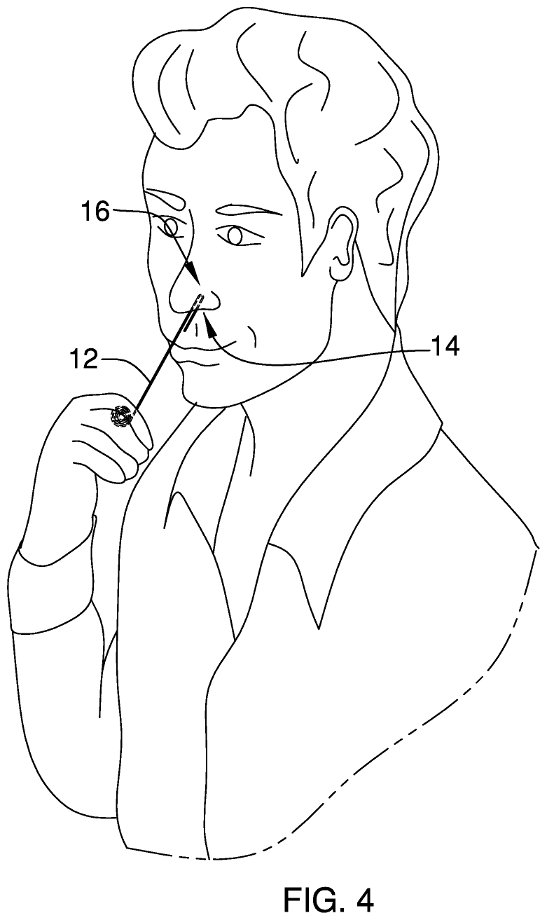

With reference now to the drawings, and in particular to FIGS. 1 through 5 thereof, a new bactericide/virucide device embodying the principles and concepts of an embodiment of the disclosure and generally designated by the reference numeral 10 will be described.

As best illustrated in FIGS. 1 through 5, the copper bactericide/virucide assembly 10 generally comprises a wire 12 that is comprised of copper thereby facilitating the wire 12 to kill bacteria and viruses on contact. The ability of dry copper to kill bacteria and viruses on contact was described in a research paper published in the February 2011 issue of Applied and Environmental Microbiology, written by C. Espirito Santo, E. W. Lam, C. G. Elowsky, D. Quaranta, D. W. Domaille, C. J. Chang and G, Grass. The wire 12 may have an AWG ranging between 10.0 and 14.0 and the wire 12 may be comprised of solid copper. The wire 12 is insertable into a nostril 14 thereby facilitating the wire 12 to contact mucous membranes 16 within the nostril 14. In this way the wire 12 can kill invasive microbes, bacteria and viruses on the mucous membranes 16. Thus, the wire 12 can inhibit the development of the common cold and other upper respiratory infections.

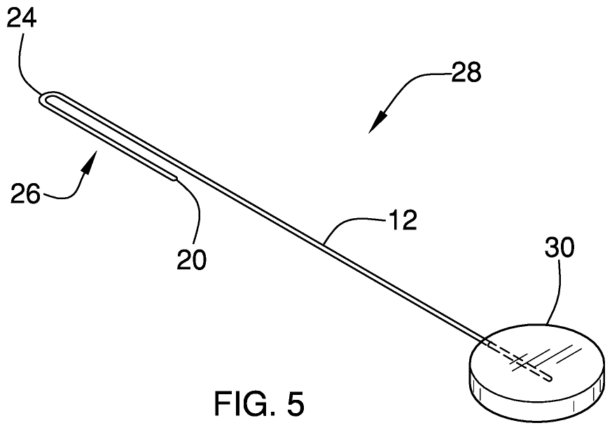

The wire 12 has a first end 18 and a second end 20, and the wire 12 is elongated between the first 18 and second 20 ends. The wire 12 is coiled around itself adjacent to the first end 18 to define a grip 22 for gripping the wire 12. Additionally, the wire 12 has a bend 24 thereon adjacent to the second end 20 to define an open loop 26 on the wire 12 having the second end 20 being directed toward the grip 22. Thus, the second end 20 resists abrading the mucous membranes 16 in the nostril 14 when the wire 12 is inserted into the nostril 14. The open loop 26 may have a length of approximately 2.0 inches. In an alternative embodiment 28 as shown in FIG. 5, a disk 30 may be coupled to the first end 18 of the wire 12 for gripping.

In use, grip 22 on the copper wire 12 is gripped and the open loop 26 is inserted into the nostril 14 when a user feels they may be developing symptoms of an upper respiratory infection. The wire 12 is manipulated to facilitate the open loop 26 to rub against all of the accessible mucous membranes 16 in the nostril 14. In this way invasive bacteria that is present in the mucous membranes 16 are exposed to the copper, thereby killing the invasive microbes, bacteria or viruses. Thus, the user can reduce the likelihood of developing the upper respiratory infection without the use of medicines or other methods of preventing upper respiratory infection.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. (canceled)

2. (canceled)

3. (canceled)

4. A copper bactericide/virucide assembly being configured to be inserted into a nostril thereby killing invasive bacteria and viruses in the nostril, said assembly comprising:
a wire being comprised of copper wherein said wire is configured to kill bacteria on contact, said wire being insertable into a nostril thereby facilitating said wire to contact mucous membranes within the nostril wherein said wire is configured to kill invasive bacteria and viruses on the mucous membranes, said wire having a first end and a second end, said wire being elongated between said first and second ends, said wire being coiled around itself adjacent to said first end to define a grip for gripping said wire, said wire having a bend thereon adjacent to said second end to define an open loop on said wire, said open loop having a straight section extending directly from said second end to said bend, said straight section being oriented parallel to said wire extending from said bend towards said first end such that said second end is directed toward said grip wherein said second end is configured to resist abrading the mucous membranes in the nostril when said wire is inserted into the nostril.
